(19) United States
(12) Patent Application Publication
(10) Pub. No.: US 2026/0319060 A1
(43) Pub. Date: Oct. 15, 2026

(54) SYSTEMS AND METHODS FOR MEDICAL ASSET TRACKING

(71) Applicant: GE Precision Healthcare LLC, Waukesha, WI (US)

(72) Inventors: Richard Inge, Richmond, VA (US); Casey Ferguson, Chesterfield, VA (US); Jason Dieter, Midlothian, VA (US)

(21) Appl. No.: 19/177,337

(22) Filed: Apr. 11, 2025

(51) Int. Cl. G01S 5/02; G06K 7/10; G06K 19/07; G16H 40/20

(52) U.S. Cl. CPC G01S 5/0294; G01S 5/0295; G06K 7/10297; G06K 19/0723; G16H 40/20

(57) ABSTRACT

Methods and systems are provided for reducing delays experienced when configuring a Real-Time Location System (RTLS) to track a tagged physical asset of a hospital. The delays may be caused by relying on a computerized maintenance management system (CMMS) for creating associations between an ID of an asset and an asset tracking tag. The delays may be avoided by creating the associations using a radio frequency identification (RFID) scanner, based on an RFID label coupled to the physical asset. An asset ID may be paired with a Bluetooth Low Energy (BLE) tag ID read by a BLE reader of the scanner. The BLE tag and the asset ID may then be transmitted from the RFID scanner to the RTLS tracking system. As a result, an amount of computational and memory resources consumed by the CMMS may be reduced.

### FIELD

Embodiments of the subject matter disclosed herein relate to medical technology inventory management, and more particularly, to tracking locations of assets in a medical environment.

### BACKGROUND

In hospitals and healthcare systems, medical assets may be managed using a Real-Time Location System (RTLS) that tracks locations of the medical assets within a medical environment. The RTLS tracking system may rely on wireless tracking tags that are physically coupled to the medical assets. In order to associate an asset with a location of a tracking tag, a data association may be formed between the tag and an asset ID using a computerized maintenance management system (CMMS). The association may be created manually using a software tool provided by the CMMS. However, due to a large number of transactions of the CMMS, the data association may be uploaded to the RTLS as part of a batch process performed periodically, such as nightly. As a result, the RTLS may not receive the data association from the CMMS promptly, leading to delays in asset tracking of new assets by 24 hours or more.

### BRIEF DESCRIPTION

In one embodiment, the issues described above may be addressed at least partly by a method for managing an inventory of physical assets, the method comprising receiving identification data of a physical asset scanned from a radio frequency identification (RFID) label coupled to the physical asset via a RFID scanner; retrieving an asset ID of the asset from a memory of the RFID scanner, based on the identification data; receiving data of an asset tracking tag coupled to the physical asset scanned via the RFID scanner, the data including a tag ID of the asset tracking tag; and uploading an association of the asset ID with the tag ID to a Real-Time Location System (RTLS). The method may be applied to monitor locations of medical devices within a hospital.

In this way, passive RFID labels may be advantageously used to pair physical assets to asset tracking tags without relying on intermediary systems, such as a computerized maintenance management system (CMMS) of the hospital, which may cause delays. Additionally, memory, processing, and network resources (e.g., bandwidth) of the CMMS may be reduced, resulting in an overall increase in efficiency of the CMMS and faster data transmission times within the hospital.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows a schematic diagram of an exemplary hospital ecosystem for tracking physical hospital assets.

FIG. 2 shows a block schematic diagram of exemplary hospital systems used for hospital asset tracking.

FIG. 3 is a first data flow diagram illustrating an exemplary flow of data during a conventional procedure for configuring an RTLS tracking system to track hospital assets, as prior art.

FIG. 4 is a second data flow diagram illustrating an exemplary flow of data during a proposed procedure for configuring the RTLS tracking system to track the hospital assets.

FIG. 5 is a flowchart illustrating a first exemplary method for generating an association between an asset tracking tag of a hospital asset and an ID of the asset in the RTLS tracking system.

FIG. 6 is a flowchart illustrating a second exemplary method for generating an association between an asset tracking tag of a hospital asset and an ID of the asset in the RTLS tracking system.

FIG. 7 is a flowchart illustrating an exemplary method for updating an RTLS tracking system with a new tagged asset.

FIG. 8 is a first exemplary timing diagram showing a timing of a first series of events occurring as a new asset is configured to be tracked in accordance with the conventional procedure of FIG. 3, as prior art;

FIG. 9 is a second exemplary timing diagram showing a timing of a second series of events occurring as a new asset is configured to be tracked in accordance with the alternative procedure of FIG. 4; and

FIG. 10 is a flowchart illustrating an exemplary method for determining a location of an asset using an RTLS tracking system.

### DETAILED DESCRIPTION

In hospitals and healthcare systems, physical medical assets may be managed using a computerized maintenance management system (CMMS), which may centralize and maintain information about the medical assets. A Real-Time Location System (RTLS) may also be used to track locations of the medical assets within a medical environment. The RTLS may rely on wireless tracking tags, such as Bluetooth Low Energy (BLE) tags, which may be physically coupled to the medical assets. The RTLS may remotely track the locations of the medical assets based on signals transmitted by the tags. For such purpose, an association between each medical asset and a corresponding tag may be recorded in the RTLS.

The RTLS may be used in conjunction with the CMMS. For example, the association between the medical assets and the corresponding tags may be created in the CMMS, and subsequently transmitted to the RTLS. In accordance with a conventional procedure, when a new asset is entered into service, a BLE tag may be attached to the new asset. The BLE tag may be associated with an ID of the new asset in the CMMS by a user of the RTLS and/or CMMS. The CMMS may transmit the association of the asset with the BLE tag to the RTLS, which may store the association in a tag table of the RTLS (e.g., in a database). After the association is stored, the RTLS may receive wireless signals from the BLE tag that indicate a location of the asset, via an infrastructure of Bluetooth-enabled receivers installed throughout a hospital environment. A technician, manager, or different user may then use the RTLS to locate or monitor the location of the asset.

However, the CMMS may have a high level of use, where computational and networking resources of the CMMS may be prioritized for non-RTLS related operations. To reduce a demand on the computational and networking resources, associations between new BLE tags and asset IDs may not be transmitted immediately or frequently from the CMMS to the RTLS. Rather, the associations may be temporarily stored, and uploaded to the RTLS in a periodic batch process. For example, associations made during the day may be uploaded to the RTLS at night. As a result, when tags are added to new assets, asset tracking of the new assets may not be immediate, where a tagged asset may not be immediately findable by staff for patient care.

To address this problem, systems and methods are proposed herein for increasing an efficiency with which physical assets are tagged and tracked by the RTLS tracking system. Rather than relying on the CMMS to generate the association between a physical asset and an asset tracking tag, the association may be advantageously created using a radio frequency identification (RFID) scanner, based on an RFID label coupled to the physical asset. Each medical asset may include an RFID label that identifies the asset. When the RFID label is scanned by the RFID scanner, the RFID scanner may look up an asset ID of the medical asset stored in a memory of the RFID scanner. The RFID scanner may include a BLE reader, and the BLE tag may be read by the BLE reader. The BLE tag and the asset ID may be associated using the RFID scanner. The association may then be transmitted from the RFID scanner to the RTLS tracking system to be stored in the tag tables of the RTLS tracking system. In contrast with the conventional procedure, the transmission of the association to the RTLS may be performed in real time, meaning without any intentional delay. In this way, the association may be generated and recorded in the RTLS tracking system without relying on the CMMS. As a result, the medical assets may be tracked by the RTLS without the delays imposed by the CMMS. In addition, an amount of computational and memory resources consumed by the CMMS may be reduced, freeing up the resources for other tasks, and networking resources consumed in communications between systems in the hospital environment may be reduced.

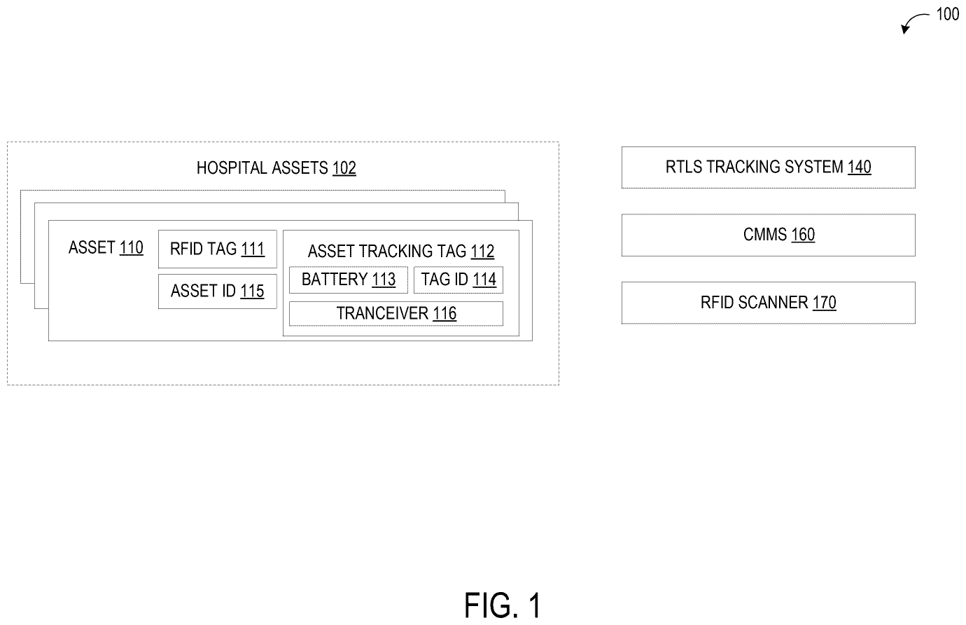

Referring now to FIG. 1, a hospital environment 100 is shown including a plurality of physical hospital assets 102, an RTLS tracking system 140, and a CMMS 160. Hospital assets 102 may include various equipment, devices, furniture, computers, scanners, etc. of varying sizes. In the depicted example, hospital assets 102 includes a plurality of assets 110, where each asset 110 includes an RFID tag 111, an asset tracking tag 112, and an asset ID 115. RFID tag 111 may be a passive RFID tag used to inventory hospital assets 102. Due to the RFID tag 111 being inexpensive (e.g., 50 cents), RFID tag 111 may be included in all hospital assets 102 as a matter of hospital policy. Asset ID 115 may be a different identifier used to identify the asset within CMMS 160. Asset tracking tag 112 may be an active tag capable of transmitting a signal to a tag-reading device. For such purpose, asset tracking tag 112 may include a transceiver 116 that generates the signal. Due to its active components, asset tracking tag 112 may be more expensive than the RFID tags (e.g., $15 each), and therefore some hospital assets 102 may lack the asset tracking tag 112. The signal may include a tag ID 114 of the asset tracking tag 112. For communications, asset tracking tag 112 may include a battery 113. Battery 113 may not be removable or rechargeable within hospital environment 100. Said in another way, battery 113 may be integrated and single use. As a result, when a charge of battery 113 decreases below a threshold charge, asset tracking tag 112 may no longer function and a replacement may be desired.

Transceiver 116 may include a short range wireless communication module for radio frequency (RF) communication with a tag reader. For the purposes of this disclosure, “short range” is less than about 10 m, as opposed to “long range”, which is more than about 100 m. In some implementations, “short range” may cover up to about 100 m, but the signal strength may be weak. Examples of wireless RF communication networks include ZigBee, Bluetooth Low Energy (BLE), WiFi (sometimes referred to as WLAN), LTE, and WiMax. In some embodiments, a CDMA/GMS communication network, which can be considered to be a cellular frequency, may be additionally or alternately used. In another embodiment, a low power WAN (LP_WAN) network or satellite may be additionally or alternately used. In the examples described herein, the asset tracking tag is a BLE tag that communicates with a BLE reader via a BLE communication protocol. However, it should be appreciated that other short range wireless communication protocols and/or networks may be used in other embodiments without departing from the scope of this disclosure.

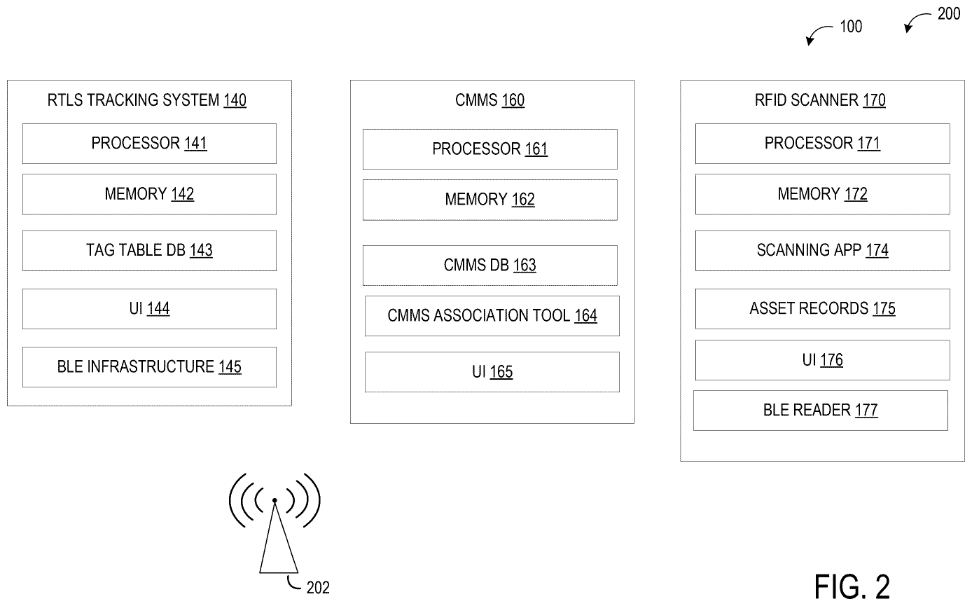

FIG. 2 shows an expanded view 200 of RTLS tracking system 140, CMMS 160, and RFID scanner 170. RTLS tracking system 140, CMMS 160, and RFID scanner 170 may communicate with each other via a wireless network 202 of hospital environment 100. Network 202 may include in a non-limiting manner, a wide area network (WAN); a local area network (LAN); the Internet; a wired or wireless (e.g. optical, Bluetooth, Bluetooth Low Energy (BLE), radio frequency (RF)) network; a cloud-based computer infrastructure of computers, routers, servers, gateways, etc., or any combination thereof associated therewith that allows one or more computing devices within the hospital environment 100 to connect with each other. Network 202 may include a public network, or a private network associated with a portion of a care facility, for example a surgery module or department of a hospital, or may be more broadly located across medical devices of an entire hospital or hospital system.

RTLS tracking system 140 includes a processor 141, a memory 142, a tag table database (DB) 143, a user interface (UI) 144, and a BLE infrastructure 145. Processor 141 may control the operation of RTLS tracking system 140 in response to instructions stored in memory 142 and/or control signals received from UI 144. In some embodiments, UI 144 may be integrated into RTLS tracking system 140, where a user may interact with, adjust, or select control elements in UI 144 (e.g., buttons, knobs, touchscreen elements, etc.) to send one or more control signals to processor 141 from UI 144. In other embodiments, the UI 144 is not integrated into RTLS tracking system 140, and the user may interact with, adjust, or select control elements in UI 144 via a user input device, such as a mouse, track ball, touchpad, etc., or the user may interact with UI 144 via a separate touchscreen, where the user touches a display screen of UI 144 to interact with UI 144, or via another type of input device. UI 144 may include a display (e.g., screen or monitor) and/or other subsystems.

Processor 141 may execute instructions stored on memory 142 to control RTLS tracking system 140. As discussed herein, memory 142 may include any non-transitory computer readable medium in which programming instructions are stored. For the purposes of this disclosure, the term “tangible computer readable medium” is expressly defined to include any type of computer readable storage. The example methods and systems may be implemented using coded instruction (e.g., computer readable instructions) stored on a non-transitory computer readable medium such as a flash memory, a read-only memory (ROM), a random-access memory (RAM), a cache, or any other storage media in which information is stored for any duration (e.g. for extended period time periods, permanently, brief instances, for temporarily buffering, and/or for caching of the information). Computer memory of computer readable storage mediums as referenced herein may include volatile and non-volatile or removable and non-removable media for a storage of electronic-formatted information such as computer readable program instructions or modules of computer readable program instructions, data, etc. that may be stand-alone or as part of a computing device. Examples of computer memory may include any other medium which can be used to store the desired electronic format of information and which can be accessed by the processor or processors or at least a portion of a computing device. In various embodiments, memory 142 may include an SD memory card, an internal and/or external hard disk, USB memory device, or similar modular memory.

RTLS tracking system 140 may track a location of a plurality of assets 110 distributed throughout hospital environment 100, based on the asset tracking tag 112 of each respective asset 110. For such purpose, tag table DB 143 may include one or more tag tables that store associations between asset tracking tags 112 and asset IDs 115 of a plurality of assets 110. When a user wishes to determine a location of an asset 110, the user may enter an asset ID 115 of the asset 110 into UI 144. RTLS tracking system 140 may retrieve a tag ID 114 of an asset tracking tag 112 of the asset 110 that is paired with the asset ID 115 from a tag table of tag table DB 143. RTLS tracking system 140 may then locate the asset 110 by determining a location of the asset tracking tag 112 using BLE infrastructure 145. BLE infrastructure 145 includes a plurality of Bluetooth-enabled tracking devices positioned throughout hospital environment 100 configured to receive radio signals transmitted periodically by the various transceivers 116 of the asset tracking tags 112 of hospital assets 102. When an asset tracking tag 112 is within a range of a tracking device, the tracking device receives a signal from the respective transceiver 116 including the asset ID 115 of the asset tracking tag 112 and location information of the asset tracking tag 112. The tracking device may transmit the location information to RTLS tracking system 140. RTLS tracking system 140 may then display the location information to the user via UI 144.

CMMS 160 includes a processor 161, a memory 162, a CMMS DB 163, a CMMS association tool 164 (e.g., a software application), and a UI 165. Processor 161 and memory 162 may be similar to processor 141 and memory 142, respectively. CMMS DB 163 may store information on each asset 110 of hospital assets 102 for asset management and maintenance purposes. The information stored may be indexed by an asset ID 115 of each asset 110. A user of CMMS 160 may look up information about an asset 110 by entering a respective asset ID 115 of the asset 110 into UI 165. CMMS 160 may retrieve the information associated with the respective asset ID 115 and display the information to the user via UI 165.

RFID scanner 170 may be used by a service technician, hospital maintenance worker, administrator, or a different user to read the RFID tags 111 included at each asset 110. RFID tags 111 may be read by RFID scanner 170 when an inventory of hospital assets 102 is performed, for example. RFID scanner 170 includes a processor 171 and a memory 172, which may be similar to processors 141 and memory 142 of RTLS tracking system 140 and/or processors 161 and memory 162 of CMMS 160. RFID scanner 170 additionally includes a scanning application 174, a plurality of stored asset records 175, a UI 176, and a BLE reader 177. Processor 171 may control the operation of RFID scanner 170 in response to instructions stored in memory 172 and/or control signals received from UI 176. Scanning application 174 may be used to scan a respective RFID tag 111 of an asset 110, in accordance with techniques and technologies known in the art. BLE reader 177 may be used to read (e.g., receive information from) a respective asset tracking tag 112 of the asset 110. Information about the asset 110, including the information received from the respective asset tracking tag 112, may be stored at asset records 175. The information stored at asset records 175 may periodically be uploaded to CMMS 160 and/or other hospital systems.

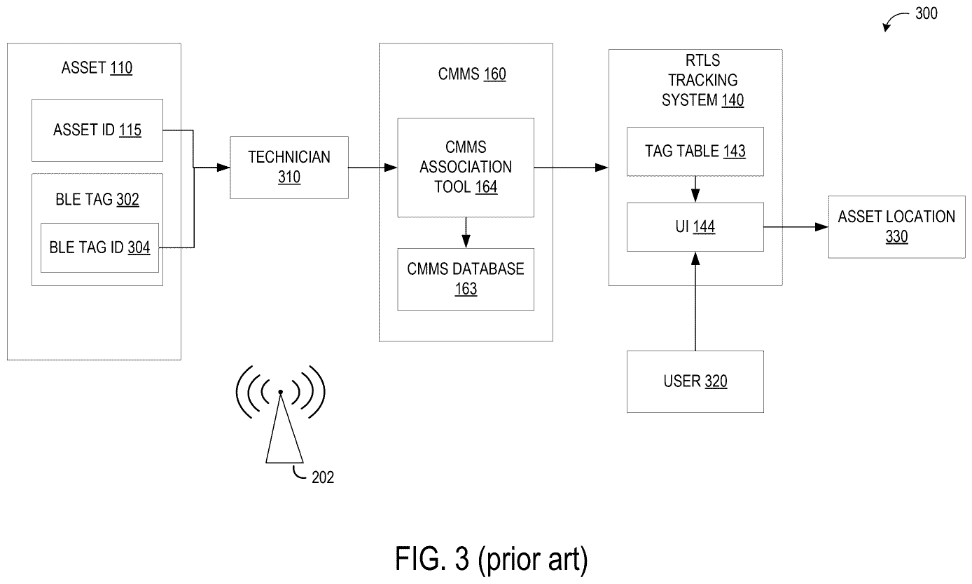

CMMS DB 163 may not store location information of an asset 110. However, in accordance with a conventional (e.g., prior art) procedure, CMMS association tool 164 may be used to pair an asset ID 115 of an asset 110 with a tag ID 114 of an asset tracking tag 112 of the asset 110. The conventional procedure is illustrated in FIG. 3.

Referring to FIG. 3, a data flow diagram 300 shows a flow of information during the introduction of a new asset 110 into hospital environment 100, in accordance with a conventional procedure. A similar flow/procedure may occur when an asset tracking tag 112 stops transmitting location information of an existing asset 110. For example, a charge of battery 113 of the asset 110 may decrease below a threshold charge for transmitting the location information, or the asset 110 may be damaged or become detached from the asset 110.

In accordance with the conventional procedure depicted in FIG. 3, a pairing of an asset ID 115 and a BLE tag ID 304 of a BLE tag 302 (e.g., tag ID 114 of asset tracking tag 112) of the new asset 110 is performed by a service technician 310. When the new asset 110 is introduced into the hospital environment, service technician 310 may physically attach BLE tag 302 to the asset 110. Service technician 310 may then open CMMS association tool 164, for example, on a laptop or tablet device of service technician 310. Service technician 310 may enter the asset ID 115 and the BLE tag ID 304 of the asset 110 into a UI of CMMS association tool 164, to create an asset ID/tag ID pairing. CMMS association tool 164 may transmit the asset ID/tag ID pairing to RTLS tracking system 140 over wireless network 202 to be stored in tag table DB 143. After the asset ID/tag ID pairing has been stored in tag table DB 143, an asset location 330 of the asset 110 may be findable by a user 320 using UI 144 of RTLS tracking system 140. User 320 may be technician 310, or an administrator of the hospital, maintenance worker, etc.

However, to reduce a consumption of processing resources of processor 161 and network resources (e.g., bandwidth) of network 202, the asset ID/tag ID pairing of the new asset 110 may not be transmitted to RTLS tracking system 140 promptly. Rather, the asset ID/tag ID pairing of the new asset 110 may be temporarily stored in CMMS DB 163. Various new asset ID/tag ID pairings may be accumulated at CMMS 160 in this manner, and then transmitted to RTLS tracking system 140 periodically, in batches. The batches may be transmitted at times when the consumption of processing resources of processor 161 and network resources of network 202 may be low, such as at night. This may generate delays in RTLS tracking system 140 receiving new asset ID/tag ID pairings, which may make it harder to determine locations of new assets.

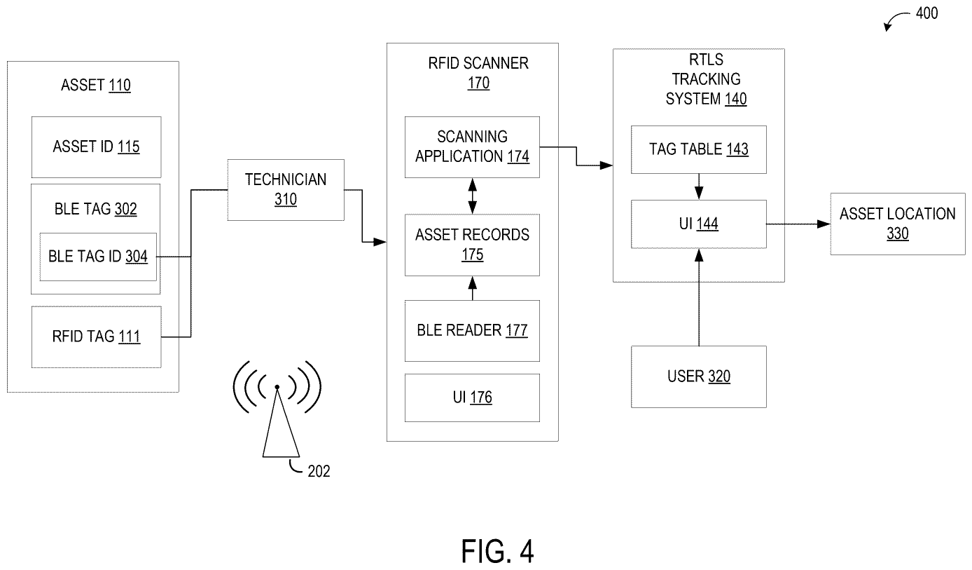

RTLS tracking system 140 may be more quickly and efficiently updated with new assets in accordance with an alternative procedure shown in FIG. 4. Referring now to FIG. 4, a proposed workflow 400 of the alternative procedure is shown. In accordance with proposed workflow 400, when the new asset 110 is introduced into hospital environment 100, service technician 310 may manually read RFID tag 111 of the new asset 110 using RFID scanner 170. Service technician 310 may scan RFID tag 111 using scanning application 174. When RFID tag 111 is received by scanning application 174, scanning application 174 may retrieve asset ID 115 of new asset 110 from asset records 175, which may include asset data previously downloaded from CMMS 160. Service technician 310 may then scan BLE tag 302 using BLE reader 177 included in an RFID scanner 170. The service technician may retrieve BLE tag ID 304 from a signal transmitted to BLE reader 177 from BLE tag 302. Service technician 310 may then pair BLE tag ID 304 with asset ID 115 via UI 176. The pairing may be performed automatically when BLE tag ID 304 is retrieved. Scanning application 174 may then transmit the asset ID/tag ID pairing created by service technician 310 to RTLS tracking system 140. When the asset ID/tag ID pairing is received at RTLS tracking system 140, the asset ID/tag ID pairing may be stored in tag table database 143. User 320 may then query RTLS tracking system 140 via UI 144 to determine asset location 330 of new asset 110.

In this way, service technician 310 advantageously bypasses CMMS 160, by creating the asset ID/tag ID pairing at RFID scanner 170. By creating the pairing on RFID scanner 170 rather than using CMMS association tool 164, the asset ID/tag ID pairing may be transmitted to RTLS tracking system 140 when BLE tag 302 is attached to the new asset 110. The location 330 of new asset 110 may then be immediately determinable by user 320 using RTLS tracking system 140. That is, user 320 may enter the asset ID 115 of new asset 110 into UI 144; RTLS tracking system 140 may retrieve the BLE tag ID 304 associated with the asset ID 115 in a tag table of the tag table database 143 based on the respective asset ID/tag ID pairing; RTLS tracking system 140 may determine the location of new asset 110 based on the BLE tag ID 304, using BLE infrastructure 145; and the location may be displayed to user 320 via UI 144.

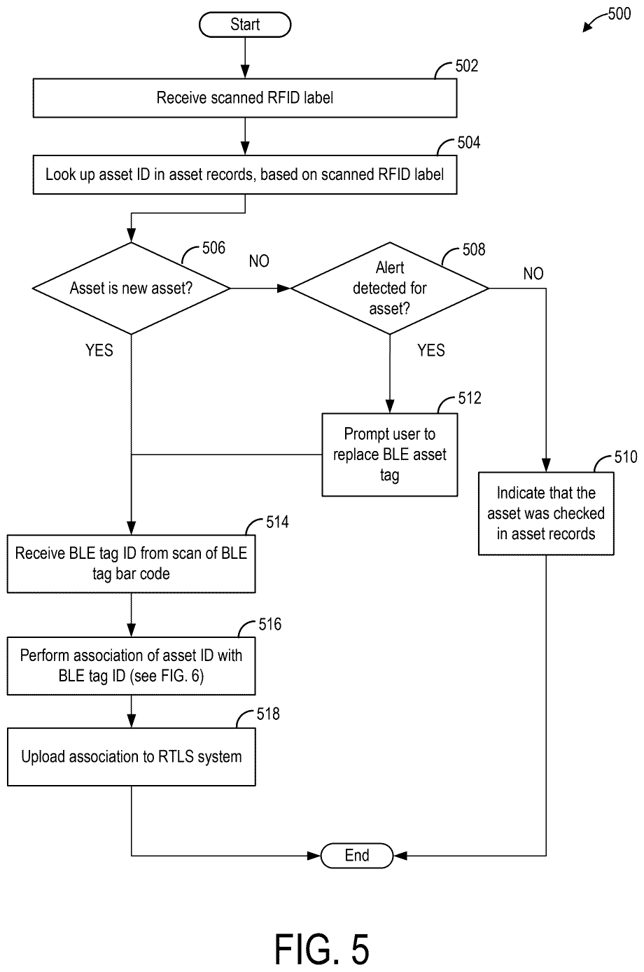

FIG. 5 shows a method 500 for performing an association (e.g., a pairing) between an asset ID of an asset of a hospital such as asset 110, and a BLE tag ID of an asset tracking tag of the asset (e.g., asset tracking tag 112 and/or BLE tag 302 of FIGS. 3 and 4), in accordance with the alternative workflow described above in reference to FIG. 4. Method 500 and the other methods described herein are described with regard to the systems and components depicted in FIGS. 1-4, though it should be appreciated that the methods may be implemented with other systems and/or components without departing from the scope of the present disclosure. Method 500 may be performed by a processor of an RFID scanner, such as processor 171 of RFID scanner 170, based on instructions stored in a memory of the RFID scanner (e.g., memory 172). Method 500 may be performed while a user of the RFID scanner, such as a service technician, introduces a new asset into a hospital environment or verifies the functioning of a BLE asset tracking tag of an existing asset within the hospital environment.

Method 500 starts at 502, where method 500 includes receiving asset identification data from a scanned RFID label of the asset. That is, the asset may have an RFID tag attached to the asset, and the user may approach a physical location of the asset, and manually scan the RFID tag with the RFID scanner.

At 504, method 500 includes looking up an asset ID (e.g., asset ID 115) of the asset in asset records (e.g., asset records 175) stored in a memory of the RFID scanner, using the asset identification data from the scanned RFID tag as a reference. The asset ID may be associated with the asset in a CMMS (CMMS 160) of the hospital, and may be used to identify the asset for management and/or maintenance purposes. For example, information about a date on which the asset was introduced into the hospital environment, a cost of the asset, a manufacturer of the asset, etc. may be stored in the CMMS using the asset ID as a reference or index. The asset records stored in the RFID scanner may be downloaded from the CMMS periodically, and may include a subset of the information stored in the CMMS.

At 506, method 500 includes determining whether the asset is a new asset or an existing asset within the hospital environment. If there is information regarding the asset in the asset records, it may be inferred that the asset is an existing asset; otherwise, it may be inferred that the asset is a new asset. If it is determined that the asset is an existing asset, method 500 proceeds to 508. If the asset is a new asset, method 500 proceeds to 514.

At 508, method 500 includes determining whether an alert is detected in the asset records for the asset. Each asset record may include alerts that indicate that an action might have to be taken. For example, an alert may be “Low Battery”, indicating that a BLE tag attached to the asset has a low battery, as determined by a last detection by an installed BLE infrastructure (e.g., BLE infrastructure 145). This alert would tell the user to apply a new BLE tag to the asset.

If at 508 an alert is detected for the asset, method 500 proceeds to 512. At 512, method 500 may include prompting the user to replace the BLE tag of the asset. Method 500 then proceeds to 514. Alternatively, if at 508 no alert is detected, it may be inferred that the BLE tag of the asset is operating within expectations, whereby method 500 proceeds to 510. At 510, method 500 includes adding an annotation to the asset records indicating that the asset was checked, and method 500 ends.

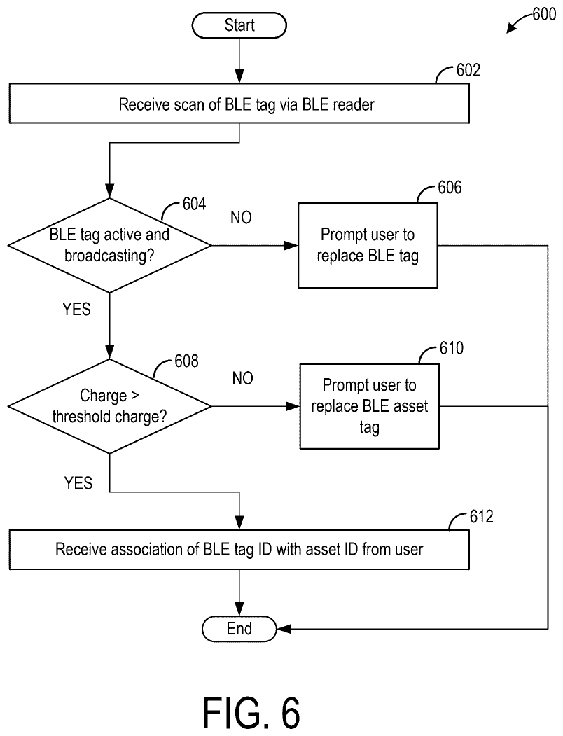

At 514, method 500 includes receiving a BLE tag ID from a scan of a bar code of a BLE tag attached to the asset. That is, after scanning the RFID tag of the asset with the RFID scanner, the user may scan the BLE tag of the asset using a BLE reader (e.g., BLE reader 177) of the RFID scanner. When the BLE tag ID is received, at 516, method 500 includes performing an association of the BLE tag ID with the asset ID retrieved from the asset records. Performing the association or pairing of the BLE tag ID and the asset ID is described in greater detail below in reference to FIG. 6.

After the association has been created at the RFID scanner, at 518, method 500 includes uploading the association to an RTLS tracking system of the hospital, where the association may be stored and used by the RTLS tracking system to track a location of the asset within the hospital environment. Method 500 ends.

Referring now to FIG. 6, a method 600 is shown for performing an association (e.g., a pairing) between an asset ID of an asset of a hospital (asset 110), and a BLE tag ID of an asset tracking tag of the asset (e.g., asset tracking tag 112 and/or BLE tag 302 of FIGS. 3 and 4), in accordance with the alternative workflow described above in reference to FIG. 4. Method 600 may be performed by a processor of an RFID scanner, such as processor 171 of RFID scanner 170, based on instructions stored in a memory of the RFID scanner (e.g., memory 172). Method 600 may be performed as part of method 500 described above in reference to FIG. 5.

Method 600 starts at 602, where method 600 includes receiving a scan of a BLE tag via a BLE reader of the RFID scanner. That is, a user of the RFID scanner may physically read the BLE tag via the RFID scanner.

At 604, method 600 includes verifying an activation of the BLE tag, and verifying that BLE message packets are being broadcasted by the BLE tag. If at 604 it is determined that the BLE tag is not active or that BLE message packets are not being broadcasted by the BLE tag (e.g., 604 is NO), method 600 proceeds to 606, where method 600 includes prompting the user to replace the BLE tag. After 606, method 600 ends.

Alternatively, if it is determined at 604 that the BLE tag is active and that BLE message packets are being broadcasted by the BLE tag (e.g., 604 is YES), method 600 proceeds to 608. At 608, method 600 includes determining whether a charge of a battery of the BLE tag is greater than a threshold charge relied on for transmitting data over a network (e.g., network 202). The charge of the battery may be included in the information received from the BLE tag when the BLE tag is scanned by the BLE reader. If at 608 it is determined that the charge of the battery is less than the threshold charge, method 600 proceeds to 610 (e.g., 606 is NO), where method 600 includes prompting the user to replace the BLE tag. After 610 method 600 ends.

Alternatively, if it is determined at 608 that the charge of the battery is greater than the threshold charge (e.g., 608 is YES), method 600 proceeds to 612.

At 612, method 600 includes receiving an association of the BLE tag ID with the asset ID retrieved in method 500 from the user. In various embodiments, the user may create the tag ID/asset ID pairing manually via a UI of the RFID scanner (e.g., UI 176). After 612, method 600 ends.

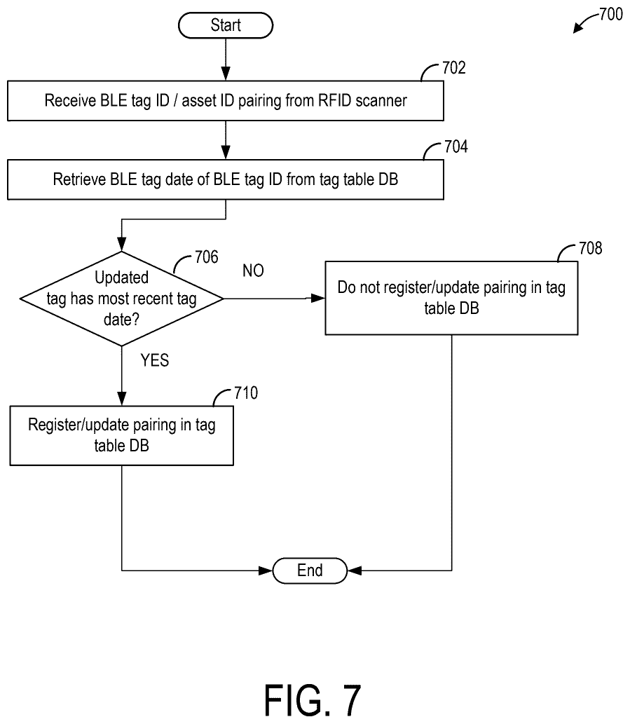

Referring now to FIG. 7, a method 700 is shown for registering an association (e.g., a pairing) between an asset ID of an asset of a hospital (asset 110), and a BLE tag ID of an asset tracking tag of the asset (e.g., asset tracking tag 112 and/or BLE tag 302 of FIGS. 3 and 4) at an RTLS tracking system, in accordance with the alternative workflow described above in reference to FIG. 4. Method 700 may be performed by a processor of the RTLS tracking system, such as processor 141 of RTLS tracking system 140, based on instructions stored in a memory of the RTLS tracking system (e.g., memory 142).

Method 700 starts at 702, where method 700 includes receiving a BLE tag ID/asset ID pairing over a network, from an RFID scanner (RFID scanner 170). The BLE tag ID/asset ID pairing may be generated by the RFID scanner by following one or more steps of method 600 of FIG. 6.

At 704, method 700 includes retrieving a BLE tag date of the BLE tag from a previous pairing of the BLE tag in a tag table database of the RTLS tracking system. As described above, the tag table database may store a plurality of associations of assets and BLE tags attached to the assets, for location tracking purposes.

At 706, method 700 includes verifying that the updated BLE tag of the asset has a more recent tag date than the BLE tag data retrieved from the previous pairing. If a first date of the updated BLE tag is not later than a second date of a previously recorded BLE tag (e.g., 706 is NO), then method 700 proceeds to 708, where the BLE tag ID/asset ID pairing received from the RFID scanner is not updated in the tag table DB of the RTLS tracking system. Alternatively, if the first date of the updated BLE tag is later than the second date of the previously recorded BLE tag (e.g., 706 is YES), or if no previously recorded pairing of the BLE tag is detected in the tag table database, then method 700 proceeds to 710.

At 710, method 700 includes registering the pairing in a tag table database of the RTLS tracking system. Registering the pairing may include registering a new pairing of a new asset introduced into the hospital environment, or updating a pairing of an existing asset within the hospital environment. For example, a pairing may exist in the tag table database for an asset ID of an asset (e.g., asset 110) and a BLE tag ID of a first BLE tag coupled to the asset. However, a service technician may detect that the first BLE tag is no longer working, and may replace the first BLE tag with a second BLE tag. When the first BLE tag is replaced, the service technician may update the pairing such that the asset ID is associated with an ID of the second BLE tag, rather than the first BLE tag. The updated pairing may be transmitted to the RTLS tracking system. After 710, method 700 ends.

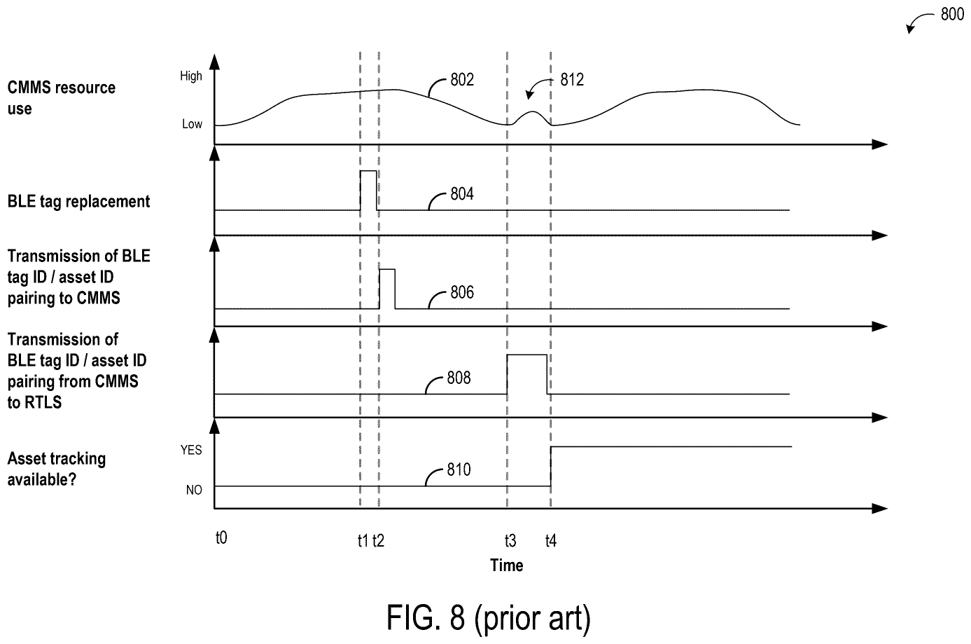

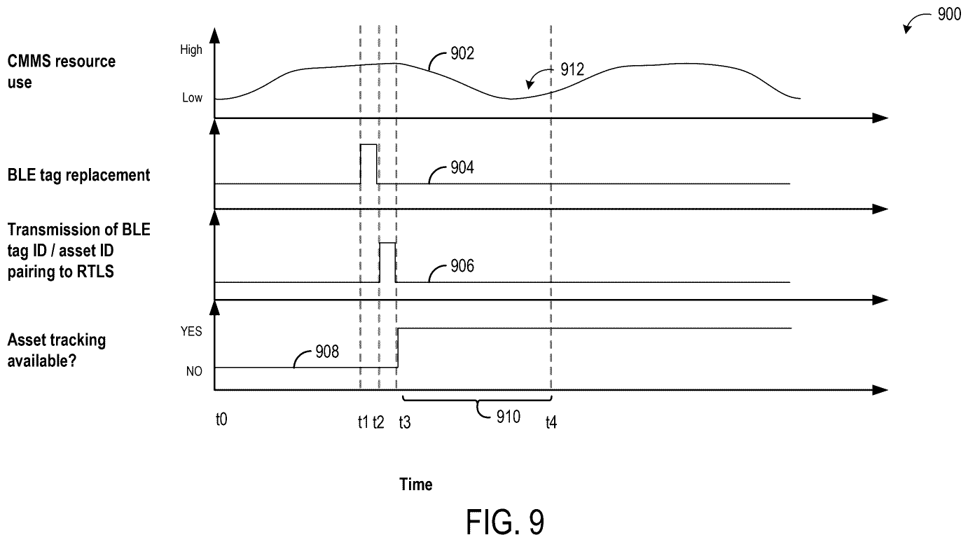

The advantage of the alternative procedure shown in FIG. 4 over the conventional procedure shown in FIG. 3 may be illustrated in FIGS. 8 and 9. Referring now to FIG. 8, a first timing diagram 800 follows the conventional procedure described in reference to FIG. 3. The first timing diagram 800 is shown that shows a timing of events occurring in a hospital as a service technician replaces a first, non-functioning BLE tag of a hospital asset with a second, functioning BLE tag, and registers a pairing of an asset ID of the asset with an ID of the BLE tag at an RTLS tracking system for asset tracking purposes. First timing diagram 800 includes five plots, where time is plotted on a horizontal axis and significant events are indicated by dashed vertical lines. A first plot 802 shows a consumption of resources over time of a CMMS of the hospital (e.g., CMMS 160). A second plot 804 indicates a time at which the BLE tag replacement occurs. A third plot 806 indicates a time at which a BLE tag ID/asset ID pairing is transmitted to the CMMS. A fourth plot 808 indicates a time at which a BLE tag ID/asset ID pairing is transmitted from the CMMS to the RTLS. A fifth plot 810 indicates when asset tracking is available for the asset using the RTLS.

At a time to, the first, non-functioning BLE tag is not broadcasting a location of the asset, and asset tracking of the asset is not available. Time to may correspond to a morning at the hospital, when CMMS resource consumption is low.

Between time t0 and time t1, the CMMS resource use increases as work is performed over the course of a first day at the hospital. At time t1, the service technician finds the non-tracked asset and detects that the first BLE tag is not functioning. The service technician replaces the first BLE tag with the second, functioning BLE tag. When the service technician replaces the first BLE tag with the second, functioning BLE tag, the service technician creates a pairing of an asset ID of the asset with an ID of the BLE tag in a software application of the CMMS (e.g., CMMS association tool 164), in accordance with the conventional procedure described in reference to FIG. 3.

At a time t2, the asset ID/tag ID pairing is transmitted to the CMMS. The transmission occurs at a middle of the first day, at a time when the CMMS and network resource use is near a maximum. As a result of the CMMS resource use being high, the asset ID/tag ID pairing is not immediately transmitted to the RTLS. Rather, a delay is imposed at the CMMS in order not to tax the CMMS resources.

At a time t3, the asset ID/tag ID pairing is transmitted to the RTLS, as part of a batch process that may include a plurality of asset ID/tag ID pairings. The batch process is performed at night, when consumption of the CMMS resources is low. However, the CMMS resource use experiences an increase between time t3 and a time t4, due to the batch process being executed, indicated at a point 812 on plot 802.

At a time t4, the asset ID/tag ID pairing is received at the RTLS, and the asset ID/tag ID pairing is stored in the tag table database of the RTLS. As a result, after time t4, asset tracking is available for the asset.

Turning now to FIG. 9, a second timing diagram 900 is shown of a timing of events occurring in a hospital as a service technician replaces a first, non-functioning BLE tag of a hospital asset with a second, functioning BLE tag, and registers a pairing of an asset ID of the asset with an ID of the BLE tag at an RTLS tracking system for asset tracking purposes, following the alternative procedure described in reference to FIG. 4. Second timing diagram 900 includes four plots, where time is plotted on a horizontal axis and significant events are indicated by dashed vertical lines. A first plot 902 shows a consumption of resources over time of a CMMS of the hospital (e.g., CMMS 160). A second plot 904 indicates a time at which the BLE tag replacement occurs. A third plot 906 indicates a time at which a BLE tag ID/asset ID pairing is transmitted to the RTLS. A fourth plot 908 indicates when asset tracking is available for the asset using the RTLS.

At a time to, the first, non-functioning BLE tag is not broadcasting a location of the asset, and asset tracking of the asset is not available. Time t0 may correspond to a morning at the hospital, when CMMS resource consumption is low.

Between time to and time t1, the CMMS resource use increases as work is performed over the course of a first day at the hospital. At time t1, the service technician finds the non-tracked asset and detects that the first BLE tag is not functioning. The service technician replaces the first BLE tag with the second, functioning BLE tag. When the service technician replaces the first BLE tag with the second, functioning BLE tag, the service technician creates a pairing of an asset ID of the asset with an ID of the BLE tag via an RFID scanner, in accordance with the alternative procedure described in reference to FIG. 4 and methods 500 and 600 of FIGS. 5 and 6, respectively.

At a time t2, the asset ID/tag ID pairing is transmitted to the RTLS. While the transmission occurs at a middle of the first day, CMMS resources are not used. Rather, the transmission is performed over the network between the RFID scanner and the RTLS without delay.

At a time t3, the asset ID/tag ID pairing is received at the RTLS, and the asset ID/tag ID pairing is stored in the tag table database of the RTLS. As a result, after time t3, asset tracking is available for the asset.

Second timing diagram 900 (e.g., the disclosed alternative procedure) may be directly compared with first timing diagram 800 (e.g., the conventional procedure). It may be noted that when the asset ID/tag ID pairing is transmitted to the RTLS via the RFID scanner (FIG. 9) rather than the CMMS (FIG. 8), the increased consumption of the CMMS resources between time t3 and time t4 of first timing diagram 800 due to the batch process being executed does not occur in second timing diagram 900, as indicated at a portion 912 of plot 902. In this way, the alternative procedure described herein may advantageously reduce resource use of the CMMS. Additionally, asset tracking for the asset becomes available earlier when the alternative procedure is used. That is, the asset may be trackable starting at time t3 of FIG. 9, rather than a time t4 in second timing diagram 900 corresponding to when the asset would have been trackable under the conventional procedure of first timing diagram 800. In other words, when the alternative procedure described herein is used, an amount of time during which the asset is trackable is greater than when the conventional procedure is used, indicated by a difference 910 between time t4 and time t3 of second timing diagram 900.

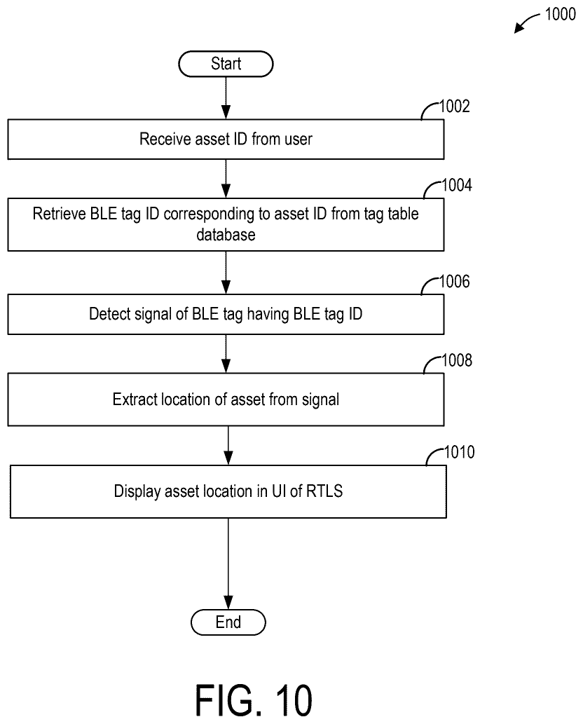

FIG. 10 shows a method for determining a location of an asset of a hospital (asset 110) at an RTLS tracking system. Method 1000 may be performed by a processor of the RTLS tracking system, such as processor 141 of RTLS tracking system 140, based on instructions stored in a memory of the RTLS tracking system (e.g., memory 142).

Method 1000 begins at 1002, where method 1000 includes receiving an asset ID from a user of the RTLS tracking system. The user may be an administrator of the hospital, or a technician, or a different type of user. The user may enter the asset ID into a UI of the RTLS tracking system (e.g., UI 144). In some examples, the asset ID may be retrieved from a database of the RTLS tracking system based on a description of the asset. For example, the user may perform a search of the asset in the database based on the description, and the asset ID may be retrieved from the database as part of the search results.

At 1004, method 1000 includes retrieving a BLE tag ID associated with the asset ID from a tag table of a tag table database (e.g., tag table database 143). The tag table database may store a plurality of asset ID/BLE tag ID pairings that are generated as described in methods 500 and 600 of FIGS. 5 and 6, respectively.

At 1006, method 1000 includes detecting a signal of a BLE tag having the BLE tag ID retrieved from the tag table database. The BLE tag may be coupled to the asset at the physical location of the asset. The signal may be periodically broadcasted by the BLE tag, and received at one or more Bluetooth-enabled devices arranged at various locations in the hospital environment (e.g., BLE infrastructure 145).

At 1008, method 1000 includes extracting a location of the asset from the signal received from the BLE tag, and at 1010, method 1000 includes displaying the asset location in the UI of the RTLS tracking system. Method 1000 ends.

Thus, systems and methods are proposed herein for increasing an efficiency of tagging and tracking assets using an RTLS tracking system. Rather than relying on a CMMS to generate the association between a physical asset and an asset tracking tag, which may impose delays due to resource consumption at the CMMS, the association may be created at an RFID scanner. When an RFID label attached to the asset is scanned by the RFID scanner, an asset ID of the asset may be retrieved from memory and paired with a BLE tag ID of a BLE tag scanned by a BLE reader of the RFID scanner. The pairing may then be transmitted from the RFID scanner to the RTLS tracking system without delay, to be stored in the tag tables of the RTLS tracking system. By not generating the pairing at the CMMS, an amount of time taken for activating asset tracking for a new asset may be reduced. The technical effect of generating the pairing at the RFID scanner rather than the CMMS is that an amount of computational and memory resources consumed by the CMMS may be reduced.

The disclosure also provides support for a method for tracking physical assets, the method comprising: receiving an asset ID of an asset at a Real-Time Location System (RTLS) tracking system, from a user of the RTLS tracking system, retrieving an asset tracking tag ID from an asset ID/tag ID pairing of the asset ID stored in a tag table database of the RTLS tracking system, detecting a signal of an asset tracking tag having the asset tracking tag ID, the asset tracking tag coupled to the asset, extracting a location of the asset from the signal, and displaying the location in a user interface (UI) of the RTLS tracking system, wherein the asset ID/tag ID pairing is uploaded to the RTLS from a radio frequency identification (RFID) scanner, the asset ID of the asset ID/tag ID pairing retrieved from a memory of the RFID scanner based on scanned RFID label coupled to the asset, the asset tracking tag ID of the asset ID/tag ID pairing received from the asset tracking tag via a tag reader of the RFID scanner. In a first example of the method, the asset tracking tag is a Bluetooth Low Energy (BLE) tag configured to transmit location data of the asset tracking tag to the RTLS, and the asset tracking tag ID is a BLE tag ID received from a BLE reader of the RFID scanner. In a second example of the method, optionally including the first example, the asset ID/tag ID pairing is uploaded to the RTLS in real time, without an intentional delay. In a third example of the method, optionally including one or both of the first and second examples, the method further comprises: prior to uploading the asset ID/tag ID pairing, verifying an activation of the BLE tag and a broadcasting of a BLE message packet from the BLE tag, and in response to detecting one of the BLE tag not being activated and the BLE message packet not being broadcasted, displaying a prompt in a UI of the RFID scanner for a user of the RFID scanner to replace the BLE tag. In a fourth example of the method, optionally including one or more or each of the first through third examples, the method further comprises: prior to uploading the asset ID/tag ID pairing, measuring a charge of a battery of the BLE tag, and in response to the measured charge being below a threshold charge, displaying a prompt in a UI of the RFID scanner for a user of the RFID scanner to replace the BLE tag. In a fifth example of the method, optionally including one or more or each of the first through fourth examples, the method further comprises: detecting an alert associated with the asset when retrieving the asset ID of the asset ID/tag ID pairing from the memory of the RFID scanner, and in response, displaying a prompt in a UI of the RFID scanner for a user of the RFID scanner to replace the BLE tag. In a sixth example of the method, optionally including one or more or each of the first through fifth examples, the method further comprises: after the asset ID/tag ID pairing is uploaded to the RTLS: detecting an existing asset ID/tag ID pairing of the asset ID, in response to a first date of the BLE tag of the existing asset ID/tag ID pairing being prior to a second date of the uploaded asset ID/tag ID pairing, replacing the existing asset ID/tag ID pairing in the tag table database with the uploaded asset ID/tag ID pairing, and in response to the first date of the BLE tag of the existing asset ID/tag ID pairing not being prior to the second date of the uploaded asset ID/tag ID pairing, not replacing the existing asset ID/tag ID pairing in the tag table database with the uploaded asset ID/tag ID pairing. In a seventh example of the method, optionally including one or more or each of the first through sixth examples, the physical assets are medical assets of a hospital. In a eighth example of the method, optionally including one or more or each of the first through seventh examples, an inventory of the medical assets is maintained in a computerized maintenance management system (CMMS) of the hospital, and the asset ID/tag ID pairing is not created at the CMMS.

The disclosure also provides support for a method for tracking physical assets, the method comprising: receiving identification data of a physical asset at a radio frequency identification (RFID) scanner, the identification data scanned from an RFID label coupled to the physical asset, retrieving an asset ID of the asset from a memory of the RFID scanner, based on the identification data, receiving a tag ID of a Bluetooth Low Energy (BLE) tag coupled to the physical asset at the RFID scanner, the tag ID scanned using a BLE tag reader of the RFID scanner, uploading a pairing of the asset ID with the tag ID to a Real-Time Location System (RTLS) tracking system, and storing the asset ID/tag ID pairing in a tag table database of the RTLS tracking system. In a first example of the method, the physical assets are assets of a hospital distributed throughout a hospital environment. In a second example of the method, optionally including the first example, the method further comprises: receiving the asset ID of the asset at the RTLS tracking system from a user of the RTLS tracking system, retrieving the tag ID of the asset from the asset ID/tag ID pairing associated with the asset ID stored in the tag table database of the RTLS tracking system, detecting a signal of the BLE tag based on the tag ID, the BLE tag coupled to the asset, extracting a location of the asset from the signal, and displaying the location in a user interface (UI) of the RTLS tracking system. In a third example of the method, optionally including one or both of the first and second examples, retrieving the asset ID of the asset from the memory of the RFID scanner further comprises detecting an alert associated with the asset in the memory, and in response, displaying a prompt in a UI of the RFID scanner for a user of the RFID scanner to replace the BLE tag. In a fourth example of the method, optionally including one or more or each of the first through third examples, receiving the tag ID of the BLE tag coupled to the physical asset at the RFID scanner using the BLE reader of the RFID scanner further comprises verifying an activation of the BLE tag and a broadcasting of a BLE message packet from the BLE tag, and in response to detecting one of the BLE tag not being activated and the BLE message packet not being broadcasted, displaying a prompt in a UI of the RFID scanner for a user of the RFID scanner to replace the BLE tag. In a fifth example of the method, optionally including one or more or each of the first through fourth examples, receiving the tag ID of the BLE tag coupled to the physical asset at the RFID scanner using the BLE reader of the RFID scanner further comprises receiving a charge of a battery of the BLE tag via the tag reader, and in response to the charge being below a threshold charge, displaying a prompt in a UI of the RFID scanner for a user of the RFID scanner to replace the BLE tag.

The disclosure also provides support for a system for tracking physical assets, the system comprising: a radio frequency identification (RFID) scanner, and a Real-Time Location System (RTLS) tracking system including a processor and a memory storing instructions that when executed, cause the processor to: receive an asset ID of an asset of the physical assets from a user of the RTLS tracking system, retrieve a tag ID from a tag table database of the RTLS tracking system, based on the asset ID, detect a signal of a Bluetooth Low Energy (BLE) tag having the tag ID, the BLE tag coupled to the asset, extract a location of the asset from the signal, and display the location of the asset in a user interface (UI) of the RTLS tracking system, wherein the tag ID is associated with the asset ID in the tag table database in an asset ID/tag ID pairing that is uploaded to the RTLS from the RFID scanner. In a first example of the system, the asset ID of the asset ID/tag ID pairing is retrieved from a second memory of the RFID scanner based on scanned RFID label coupled to the asset. In a second example of the system, optionally including the first example, the tag ID of the asset ID/tag ID pairing is scanned from the BLE tag via a tag reader of the RFID scanner. In a third example of the system, optionally including one or both of the first and second examples, the RFID scanner includes a second processor and the second memory stores instructions that when executed, cause the second processor to: detect an alert associated with the asset in the second memory, and in response, display a prompt in a UI of the RFID scanner for a user of the RFID scanner to replace the BLE tag. In a fourth example of the system, optionally including one or more or each of the first through third examples, further instructions are stored in the second memory that when executed, cause the second processor to: verify an activation of the BLE tag, a broadcasting of a BLE message packet from the BLE tag, and a charge of a battery of the BLE tag, and detect one of the BLE tag not being activated, the BLE message packet not being broadcasted, and the charge of the battery being below a threshold charge, and in response, display a prompt in a UI of the RFID scanner for a user of the RFID scanner to replace the BLE tag.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method for tracking physical assets, the method comprising:
receiving an asset ID of an asset at a Real-Time Location System (RTLS) tracking system, from a user of the RTLS tracking system;
retrieving an asset tracking tag ID from an asset ID/tag ID pairing of the asset ID stored in a tag table database of the RTLS tracking system;
detecting a signal of an asset tracking tag having the asset tracking tag ID, the asset tracking tag coupled to the asset;
extracting a location of the asset from the signal; displaying the location in a user interface (UI) of the RTLS tracking system;
wherein the asset ID/tag ID pairing is uploaded to the RTLS from a radio frequency identification (RFID) scanner, the asset ID of the asset ID/tag ID pairing retrieved from a memory of the RFID scanner based on scanned RFID label coupled to the asset, the asset tracking tag ID of the asset ID/tag ID pairing received from the asset tracking tag via a tag reader of the RFID scanner, and wherein the asset ID is associated with the asset in the RTLS tracking system; and
wherein the asset tracking tag is a Bluetooth Low Energy (BLE) tag configured to transmit location data of the asset tracking tag to the RTLS, and the asset tracking tag ID is a BLE tag ID received from a BLE reader of the RFID scanner and prior to uploading the asset ID/tag ID pairing, verifying an activation of the BLE tag and a broadcasting of a BLE message packet from the BLE tag, and in response to detecting one of the BLE tag not being activated and the BLE message packet not being broadcasted, displaying a prompt in a UI of the RFID scanner for a user of the RFID scanner to replace the BLE tag.

2. (canceled)

3. The method of claim 1, wherein the asset ID/tag ID pairing is uploaded to the RTLS in real time, without an intentional delay.

4. (canceled)

5. The method of claim 1, further comprising:
prior to uploading the asset ID/tag ID pairing, measuring a charge of a battery of the BLE tag, and in response to the measured charge being below a threshold charge, displaying the prompt in the UI of the RFID scanner for the user of the RFID scanner to replace the BLE tag.

6. The method of claim 1, further comprising:
detecting an alert associated with the asset when retrieving the asset ID of the asset ID/tag ID pairing from the memory of the RFID scanner, and in response, displaying the prompt in the UI of the RFID scanner for the user of the RFID scanner to replace the BLE tag.

7. The method of claim 1, further comprising:
after the asset ID/tag ID pairing is uploaded to the RTLS:
detecting an existing asset ID/tag ID pairing of the asset ID;
in response to a first date of the BLE tag of the existing asset ID/tag ID pairing being prior to a second date of the uploaded asset ID/tag ID pairing, replacing the existing asset ID/tag ID pairing in the tag table database with the uploaded asset ID/tag ID pairing; and
in response to the first date of the BLE tag of the existing asset ID/tag ID pairing not being prior to the second date of the uploaded asset ID/tag ID pairing, not replacing the existing asset ID/tag ID pairing in the tag table database with the uploaded asset ID/tag ID pairing.

8. The method of claim 1, wherein the physical assets are medical assets of a hospital.

9. The method of claim 8, wherein an inventory of the medical assets is maintained in a computerized maintenance management system (CMMS) of the hospital, and the asset ID/tag ID pairing is not created at the CMMS.

10. A method for tracking physical assets, the method comprising:
receiving identification data of a physical asset at a radio frequency identification (RFID) scanner, the identification data scanned from an RFID label coupled to the physical asset;
retrieving an asset ID of the asset from a memory of the RFID scanner, based on the identification data;
receiving a tag ID of a Bluetooth Low Energy (BLE) tag coupled to the physical asset at the RFID scanner, the tag ID scanned using a BLE reader of the RFID scanner;
uploading a pairing of the asset ID with the tag ID to a Real-Time Location System (RTLS) tracking system;
storing the asset ID/tag ID pairing in a tag table database of the RTLS tracking system, and wherein the asset ID is associated with the asset in the RTLS tracking system; and
detecting an alert associated with the asset in the memory, and in response, displaying a prompt in a UI of the RFID scanner for a user of the RFID scanner to replace the BLE tag.

11. The method of claim 10, wherein the physical assets are assets of a hospital distributed throughout a hospital environment.

12. The method of claim 10, further comprising:
receiving the asset ID of the asset at the RTLS tracking system from a user of the RTLS tracking system;
retrieving the tag ID of the asset from the asset ID/tag ID pairing associated with the asset ID stored in the tag table database of the RTLS tracking system;
detecting a signal of the BLE tag based on the tag ID, the BLE tag coupled to the asset;
extracting a location of the asset from the signal; and
displaying the location in a user interface (UI) of the RTLS tracking system.

13. (canceled)

14. The method of claim 10, wherein receiving the tag ID of the BLE tag coupled to the physical asset at the RFID scanner using the BLE reader of the RFID scanner further comprises verifying an activation of the BLE tag and a broadcasting of a BLE message packet from the BLE tag, and in response to detecting one of the BLE tag not being activated and the BLE message packet not being broadcasted, displaying the prompt in the UI of the RFID scanner for the user of the RFID scanner to replace the BLE tag.

15. The method of claim 10, wherein receiving the tag ID of the BLE tag coupled to the physical asset at the RFID scanner using the BLE reader of the RFID scanner further comprises receiving a charge of a battery of the BLE tag via the tag reader, and in response to the charge being below a threshold charge, displaying the prompt in the UI of the RFID scanner for the user of the RFID scanner to replace the BLE tag.

16. A system for tracking physical assets, the system comprising:
a radio frequency identification (RFID) scanner; and
a Real-Time Location System (RTLS) tracking system including a processor and a memory storing instructions that when executed, cause the processor to:
receive an asset ID of an asset of the physical assets from a user of the RTLS tracking system, and wherein the asset ID is associated with the asset in the RTLS tracking system;
retrieve a tag ID from a tag table database of the memory, based on the asset ID;
detect a signal of a Bluetooth Low Energy (BLE) tag having the tag ID, the BLE tag coupled to the asset;
extract a location of the asset from the signal; and
display the location of the asset in a user interface (UI) of the RTLS tracking system;
wherein the tag ID is associated with the asset ID in the tag table database in an asset ID/tag ID pairing that is uploaded to the RTLS from the RFID scanner, and wherein the asset ID of the asset ID/tag ID pairing is retrieved from a second memory of the RFID scanner based on scanned RFID label coupled to the asset and the RFID scanner includes a second processor and the second memory stores instructions that, when executed, cause the second processor to:
detect an alert associated with the asset in the second memory, and in response, display a prompt in a UI of the RFID scanner for a user of the RFID scanner to replace the BLE tag.

17. (canceled)

18. The system of claim 16, wherein the tag ID of the asset ID/tag ID pairing is scanned from the BLE tag via a tag reader of the RFID scanner.

19. (canceled)

20. The system of claim 16, wherein further instructions are stored in the second memory that when executed, cause the second processor to:
verify an activation of the BLE tag, a broadcasting of a BLE message packet from the BLE tag, and a charge of a battery of the BLE tag; and
detect one of the BLE tag not being activated, the BLE message packet not being broadcasted, and the charge of the battery being below a threshold charge, and in response, display the prompt in the UI of the RFID scanner for the user of the RFID scanner to replace the BLE tag.
